(19) United States
(12) Patent Application Publication
(10) Pub. No.: US 2007/0122904 A1
(43) Pub. Date: May 31, 2007

(54) Method and apparatus for culturing cells

(75) Inventor: Robert E. Nordon, New South Wales (AU)

Correspondence Address: NIXON & VANDERHYE, PC, 901 NORTH GLEBE ROAD, 11TH FLOOR, ARLINGTON, VA, 22203, US

(73) Assignee: Unisearch Limited, Sydney (AU)

(21) Appl. No.: 11/646,256

(22) Filed: Dec. 28, 2006

Related U.S. Application Data
(63) Continuation of application No. 10/089,153, filed on Sep. 4, 2002.

(51) Int. Cl. C12N 15/867; C12N 5/08; C12M 3/00

(52) U.S. Cl. 435/325; 435/372; 435/455; 435/456; 435/289.1

(57) ABSTRACT

A method for culturing cells, the method comprising: providing a plurality of cellulose hollow fibre capillaries having cells and at least one protein required for proliferation, differentiation and/or genetic modification of the cells therein and optionally at least one metabolite; and providing on the extracapillary side of the semi-permeable substrate at least one metabolite required for proliferation of the cells.

### FIELD OF THE INVENTION

The present invention relates to a method and apparatus for culturing cells. The invention is particularly concerned with a method and apparatus for growing and maintaining cells in vitro at high cell densities, and for the use of such cells for therapy or in the production of engineered proteins and viruses and for biosynthesis and degradation of compounds.

### BACKGROUND

Cell culturing is important for cell biology and immunological studies and for use in medical therapies such as cell therapy. Particular examples of cell therapy include blood stem cell transplantation to regenerate blood production after high dose therapy for cancer; cellular immunotherapy to eliminate residual cancer cells or reconstitute immunity to viruses; and somatic gene therapy as a cure for genetic and viral diseases (e.g. Haemophilia, HIV).

One immediate application of cell expansion technologies is the ex vivo culture and expansion of CD34⁺ cells to abrogate low white cell counts (neutropenia and thrombocytopenia) following high dose chemotherapy and stem cell transplant for cancer (leukemia) and gene therapy. The cost to healthcare providers for hospitalisation after stem cell transplant is $A40,000 per patient. The application of stem cell transplant is growing at the rate of 10% per annum because severe complications such as transplant rejection and graft versus host disease are prevented. The main reason for patients requiring hospitalisation after stem cell transplant is related to neutropenia and thrombocytopenia which cause mucositis (break down of mucus membranes), septicaemia and bleeding. Neutropenia and thrombocytopenia is prolonged for cord stem cell transplants (1 month and 3 months respectively in children).

Initial studies have shown the administration of large numbers of neutrophil precursors generated in vitro from CD34⁺ cells using haematopoietic growth factors (G-CSF, SCF, Flt-3 ligand and thrombopoietin) can prevent neutropenia. Conventionally cells have been grown in gas-permeable bags (eg Baxter, American Flurosil).

It has become apparent that at least 5×10⁹ cells are required for an adult patient. Conventional techniques such as Flask or bag tissue culture are relatively wasteful. Using these techniques, mammalian cells grow to a maximum density of 1-2×10⁶ cells/ml. At this cell concentration, the media must be replenished because glucose depletion and lactate accumulation inhibit cellular metabolism. The process is also wasteful since proteins are discarded even though their levels are not depleted. The current cost of serum free media that is suitable for use in clinical trials is at least $A2000-$3000 per litre; the major proportion of the cost relates to manufacture of clinical grade human albumin, low density lipoproteins and recombinant growth factors. Thus for clinical applications which require transplants of up to 10¹⁰ cells, the cost of media alone is prohibitively expensive.

There is a need for more cost-effective technologies for the generation of large numbers of cells. In U.S. Pat. No. 5,763,194, the disclosure of which is incorporated herein by reference, we describe a cell separation device and method based on the use of a semi-permeable substrate in the form of an array of hollow fibres provided internally with a ligand reactive with the desired cell type. U.S. Pat. No. 5,763,194 also discloses the use of the cell separation device for cell expansion. We have carried out further research based on the hypothesis that cells will grow at high density in culture systems that maintain metabolites, such as glucose and lactate, within their physiological ranges and in which there is recycling or retention of protein components. Recycling or retention of protein components provides a high density culture system that is potentially more cost-effective.

In particular, the present inventors have found that by appropriate selection of the permeability of a semi-permeable substrate, cells captured and grown inside the semi-permeable substrate can be grown to concentrations up to 40-50 times higher than the concentration of cells that can be supported in conventional culture systems (T flasks or Teflon bags).

### DISCLOSURE OF THE INVENTION

In a first aspect, the present invention provides a method for culturing one or more type(s) of cells, the method comprising:

providing a semi-permeable substrate having the cells on one side thereof (cellular side), wherein the semi-permeable substrate is permeable to at least one substance selected from the group consisting of a nutrient, a regulator and a metabolite, but is substantially impermeable to at least one protein required for proliferation, differentiation and/or genetic modification of the cells;

contacting the cells with a culture medium comprising at least one protein required for proliferation, differentiation and/or genetic modification of the cells, and optionally at least one substance required for the proliferation of the cells; and

providing on the acellular side of the semi-permeable substrate at least one substance required for proliferation of the cells.

The cells may be unattached or, as described below, immobilised on the semi-permeable substrate.

In a preferred form of the present invention, the at least one acellular substance is contained in media flowing (perfusing) over at least a part of the acellular surface of the semipermeable membrane. In a particularly preferred form of the invention, the acellular media is recirculated to the semipermeable substrate. The acellular media perfusion rate is preferably responsive to the cellular biomass. The biomass may be determined by any suitable means, for example, by measuring oxygen uptake, glucose uptake and/or lactate output in the cellular media. Preferably the perfusion rate is controlled so as to prevent significant depletion or accumulation of one or more of these components downstream from the bioreactor. The media that is circulated on the acellular side may be replenished continuously or batchwise to prevent upstream depletion or accumulation of one or more of the components.

The at least one protein required for proliferation, differentiation and/or genetic modification of the cells may be contained in stationary media or it may be contained in media perfusing the cellular space.

The at least one substance selected from a nutrient, regulator or metabolite to which the semi-permeable substrate is permeable may be any substance other than a preselected protein or other larger molecule, required for cell proliferation, differentiation or genetic modification. The at least one substance may be any substance that is used by the cells as a metabolite or anabolite. It may be a cofactor or regulator of metabolism. The at least one substance may be a catabolite produced by metabolic breakdown by the cells.

The at least one substance may be any substance that is consumed by the cells, for example, glucose, an amino acid, a vitamin, or oxygen. The at least one substance may be low molecular weight proliferative or differentiation factor, for example, a steroid, nitric oxide etc.

The at least one substance required for proliferation of the cells may be any substance required for cell proliferation. It may be a nutrient, regulator or a substance required by the cell for metabolism.

The semi-permeable substrate may be impermeable to molecules having a molecular weight at least about 10,000 preferably, a molecular weight of at least 8,000 and most preferably, a molecular weight of a least about 5,000. The semi-permeable substrate not only allows at least one acellular substance to pass from the acellular side to the cellular side of the substrate for use by the cells, but also allows low molecular weight waste products (eg lactate) generated by the cells to pass through to the acellular side of the substrate.

In a particularly preferred embodiment, the semi-permeable substrate is in the form of at least one hollow fibre or capillary. Preferably the hollow fibres have a radius in the range of about 100 to 400 microns and a wall thickness in the range of about 6 to 50 μm. A wall thickness of about 7 μm is particularly preferred. By use of such semi-permeable hollow fibres, it is possible to maintain glucose, lactate and other metabolites within physiological range by perfusion of media containing these low molecular weight substrates on the extracapillary side. It is not necessary to supplement extracapillary media with the same proteins required for cell growth therefore resulting in substantial reduction in the amount consumed. Furthermore, because the consumption of these proteins is low, it is not always necessary to perfuse the inside of fibres with media.

Preferably the hollow fibres are formed from cellulose. Other types of hollow fibres may also be used comprising dialysis and ultrafiltration hollow fibre membranes made of cellulose acetate or polysulfone. When such ultrafiltration membranes are used, it will typically be necessary to use the membranes under conditions such that the permeability of the membrane is selective for metabolites.

Since it is possible that peptides having a molecular weight below 10,000 (eg insulin) will cross the membrane, it may be necessary to include that molecule in the acellular media. It may also be necessary to equalise the osmotic pressure caused by molecules greater than 10,000 molecular weight to prevent influx of water across the semi-permeable substrate into the cellular media. This can be achieved in a number of ways. First, by closure of valves that regulate media flow into and out of the cellular compartment, by selection of the pressure on the acellular side that is equal and opposite to the osmotic pressure or including in the acellular media, a molecule that does not cross the cellulose membrane. We have found that molecules significantly less expensive than the proteins used for cell proliferation and growth in the intra-cellular media can be used for this purpose. For example cheaper molecules such as serum albumin (BSA) or dextran (therapeutic grade, molecular weight 70,000) may be included in the acellular media to equalise the osmotic pressure across the semi-permeable substrate. The use of pressure or a molecule such as BSA or dextran on the acellular side of the substrate provides a process that is significantly less expensive than current tissue culture techniques.

As already mentioned, the cells may be immobilised on one side of the semi-permeable membrane. Preferably the cells are bound to the semi-permeable substrate by one or more ligands. The ligand may be selected from the group consisting of an antibody, lectin, growth factor and receptor. The ligand may be a monoclonal antibody. The ligand may be a cellulose binding domain chimaeric molecule.

The method of the invention may be used to culture one cell type or it may be used to culture two or more types of cells. The cells may be in the form of a coculture.

The cells may be selected from one or more animal cells, plant cells, fungi cells or microorganisms. The cells are preferably mammalian cells, although the method of the present invention may be used to expand other types of cells, for example, insect cells, plant cells, yeast or bacteria. The cells may be any cell type that is modified by genetic engineering.

Examples of mammalian cells that may be expanded using the method of the invention include, but are not limited to, haematopoietic cells (CD34⁺), T cells, B cells, dendritic cells, liver cells, bone marrow cells, pancreatic islet cells, embryonic stem cells or genetically modified cells such as chinese hamster ovary (CHO) cells or hybridomas.

The at least one protein required for cell proliferation, differentiation and/or genetic modification may be selected from one or more of the group consisting of growth factors, colony stimulating factors, cytokines, cytokine receptors, chemokines, albumin, transferrin, low density lipoproteins, and gene transfer vectors. Examples of growth factors are IL-1, IL-2, IL3, SCF, IL-6, Flt-3 ligand, insulin, thrombopoietin, erythropoietin, EGF, TNF, TGFβ, PDGF, NGF, FGF, etc. Examples of colony stimulating factors are GCSF and GMCSF. Examples of chemokines are MIP1α, SDF-1 and insulin-like growth factor. Examples of gene transfer vectors are non-replicative retroviral and adeno-associated viral vectors, lipoplexes and phage vectors. Preferably the molecule or multimolecular complex is present in excess so that it is not significantly depleted by cell consumption or biodegradation. This will vary depending on the specific molecule and the type of cells in culture. The albumin may be present in concentration in the range of about 10 to 50 mg/ml, preferably about 10 mg/ml.

The acellular media may also contain molecule(s) that scavenge waste products generated by the cells. Preferably, the semi-permeable substrate is impermeable to these scavenger molecule(s). The scavenger molecule may be a protein. Particularly preferred scavenger molecules are those reducing oxygen radical damage. Oxygen radicals are generated by the interaction of light and oxygen with hydrophobic amino acids and lipids within the culture media. These oxidise unsaturated lipids as well as stress cellular metabolism. Free radical scavengers such as Vitamin E, albumin, and mercaptoethanol may be used as scavengers.

The acellular media may also contain a buffer system to maintain the acellular media at a preselected pH. The pH may be regulated using a CO₂/bicarbonate and HEPES buffer system.

Preferably the oxygen and carbon dioxide content of the acellular media is controlled. This may be achieved by gas exchange, for example, by gas exchange across a silicone membrane.

Preferably, the acellular media is maintained at a preselected temperature. Preferably the preselected temperature is the range of about 30 to 40° C., more preferably 37° C. The method of the invention may be under computer control.

The method of the first aspect of the invention may include a preliminary step of separating a desired cell type from sample containing cells of more than one cell type. The separation step may be achieved by immobilising the desired cell type on a semi-permeable substrate and treating the semi-permeable substrate such that the cells not bound to the substrate are removed. The preliminary separation step may be carried out in a separate apparatus, for example the cell separation apparatus described in U.S. Pat. No. 5,763,194. In this embodiment, the separation step may be carried out in the same apparatus as that used to carry out the cell culturing method of the first aspect of the present invention.

In a second aspect, the present invention provides a bioreactor for the proliferation and growth of cells including a semi-permeable membrane wherein said semi-permeable substrate is permeable to at least one substance selected from the group consisting of a nutrient, a regulator and a metabolite but is substantially impermeable to at least one protein required for proliferation, differentiation and/or genetic modification of said cells and is in the form of an array of hollow fibres.

In another embodiment, the second aspect is also capable of separation of specific cells.

The semi-permeable substrate is preferably impermeable to molecules having a molecular weight greater than or equal to about 10,000, more preferably greater than or equal to 8,000. A cut-off molecular weight of 5,000 is particularly preferred.

In a particularly preferred embodiment of the bioreactor of the present invention, the hollow fibres are cellulose hollow fibres. As mentioned above, other types of hollow fibres may also be used including ultrafiltration hollow fibre membranes made of polysulfone.

In a further preferred embodiment of the second aspect of the invention, the hollow fibres are located within a housing for containing nutrient media to produce a hollow fibre module. The housing may be cylindrical. The housing may have an inlet and outlet port through which extracapillary nutrient media may be introduced and withdrawn. The housing may include an inlet and outlet port through which intracapillary media may be introduced and withdrawn. The hollow fibre module may be of a standard dialyser configuration.

Preferably the housing is in fluid communication with supply means for supplying extracapillary nutrition media and supply means for suppling intracapillary culture media. The fluid flow to the housing module may be achieved by use of pump means.

The bioreactor of the invention may include one or more gas and heat exchangers through which the media, particularly the acellular media, flows.

The bioreactor of the second aspect of the invention may include means for controlling environmental variable(s) for media contained in the supply means, for example, temperature and CO₂ concentration.

Preferably the various operations of the bioreactor of the invention are under computer control.

In yet another embodiment of the bioreactor of the invention, the hollow fibres are provided internally with a ligand reactive to the cells Examples of suitable ligands for use in the bioreactor of the invention are described above.

The bioreactor of the invention is easily scaled-up. For example two or more hollow fibre modules may be used to provide the requisite number of cells during, for example, cell therapy.

The bioreactor of the invention may dimensioned for portability so that it may be used as a patient specific bioreactor.

The method and bioreactor of the present invention have applications for cell therapy and biotechnology. Examples or these applications are:
1. Cell Expansion
2. Production of engineered proteins or viruses
3. Biosynthesis or biodegradation.
   Cell Expansion

As already mentioned above, cell expansion is defined as the process of cell proliferation by DNA replication and cell division. Cell expansion can be associated with cellular differentiation (change in phenotype) which is governed by the constituents of the cell culture media (growth factor levels and other factors). The applications where cell expansion is required are summarised below.

The main clinical application being developed is the ex vivo expansion of neutrophil and platelet precursors. These are required to prevent the prolonged period of neutropenia and thrombocytopenia that follows high dose chemoradiotherapy and haematopoietic stem cell transplant. Neutrophil and platelet precursors have been generated in vitro by stimulating haematopoietic stem cells (CD34⁺ ) to proliferate and differentiate with haematopoietic growth factors. The duration of low white cell counts (neutropenia and thrombocytopenia) following myeloablative therapies is shortened or abrogated by infusing large numbers of ex vivo generated haematopoietic cells with the stem cell transplant.

Alternatively, a coculture system may be used to generate haematopoietic cells. Here a bone marrow stromal cell layer is established that supports the growth of haematopoietic stem cells (Dexter culture system). This process could be established inside a hollow fibre module.

Cellular immunotherapy is the adoptive transfer of immune cells to treat infectious and malignant diseases. Cytotoxic T cells, which orchestrate the elimination of malignant or virus-infected tissues, are generated by T cell receptor engagement and crosslinking. Other stimulatory molecules required to drive this process are the cytokine IL-2 and engagement of accessory molecules on the T cell (CD28) by B7-1 and B7-2 molecules found on antigen presenting cell.

The role of antigen presenting cells (APCs) is to digest tumour cells or viruses into peptide antigens that can be presented bound to MHC (major histocompatibility complex) to a complementary T cell receptor on specific T cell clones. This interaction is a “lock and key” fit and requires selection of a T cell clone from a polyclonal T cell population. Once selected and expanded, the so-called antigen-specific T cells are sensitised, and have greater potency to eliminate tumour or virus infected cells

The process of selection and expansion of antigen-specific T cell clones has been achieved in vitro using a coculture system. A monolayer of APCs—dendritic cells, monocytes or fibroblasts—present peptide antigens to polyclonal T cells. Only those T cell clones that bind via the T cell receptor to the MHC-preserrted peptide on APCs are selected to expand with IL-2.

This approach has been used to reconstitute immunity to viruses such as HIV and CMV. APCs can be generated in vitro from CD34⁺ cells using cytokines.

The bioreactor may be used for the transduction of haematopoietic or immune cells by retroviral gene transfer vectors used in gene therapy. Cell expansion is required for the transgene to be stably inserted into the genome of the cell. Cells are grown at high density and do not require targe volumes of retroviral supernatant.

Production of Engineered Proteins or Viruses

In this application the cell type is genetically modified to secrete biological molecules that have biotechnological or pharmaceutical applications. Typically a cellular expression system is used to produce a protein, peptide or viral vector that is encoded by genes isolated from another organism. Examples of proteins that have been expressed in cell lines include monoclonal antibodies or their fragments, specific binding domains, enzymes, cytokines, growth factors, cell surface receptors and so on. More than one domain may be combined to form a chimaeric protein which is expressed by the genetically modified cell line. For example, replication-deficient retroviruses used for human gene therapy have been produced by modified mouse fibroblast cells. Mammalian or yeast cell lines may be used to express proteins that require synthesis of additional carbohydrate side-chains for full biological activity.

Biosynthesis and Biodegradation

The unique metabolic pathways that have developed in some organisms have been used to synthesize or degrade organic compounds. A simple example is the use of yeast fermentation to generate alcohol from carbohydrates and sugars. The degradative pathways of bacteria have been used to break down nitrogen containing organic compounds,

The invention will now be described with reference to the following non-limiting embodiments.

### BRIEF DESCRIPTION OF THE DRAWINGS

FIG. 1 is a graph showing the effect of cell attachment on growth of CD34⁺ cells (mean±SEM);

FIG. 2 is a graph showing the effect of intracapillary growth factor concentration (mean±SEM)**p<0.005

FIG. 3 are microphotographs of single hollow fibres showing the effect of intracapillary growth factor;

FIG. 4 is a graph showing the influence of insulin, BSA and dextran in extracapillary media (mean±SEM) *p<0.05, **p<0.01;

FIG. 5 is a schematic drawing one embodiment of a bioreactor in accordance with the invention;

FIG. 6 is a schematic drawing of a further embodiment of a bioreactor in accordance with the invention;

FIG. 7 is a graph showing glucose and lactate levels in EC media during a bioreactor run described in Example 4; and

FIG. 8 is a graph showing EC oxygen concentration, pH at the outlet of the hollow fibre module and EC flow rate during a bioreactor run dscribed in Example 4.

### EMBODIMENTS OF THE INVENTION

### EXAMPLE 1

Feasibility of High Density Culture using Cellulose Hollow Fibres

High-density bioreactors provide a technology for production of mammalian cells or their products using a compact configuration. Another potential benefit of high-density culture is the reduced consumption of expensive or scarce media components such as human albumin, retroviral supernatant or growth factors.

The following experiments establish the feasibility of high-density culture of haematopoietic cells using cellulose hollow fibres.

Aims

1. To establish the final concentration cells will reach when grown inside cellulose hollow fibres given an excess of media in the extracapillary space
2. To establish which factors limit the growth of cord blood CD34⁺ cells inside cellulose hollow fibres.
3. To minimise the consumption of expensive components (growth factors and albumin) using the cellulose hollow fibre culture system.
4. Determine optimal extra-and intracapillary media composition for expansion of cord blood CD34⁺ cells using the device.
   Methods
   Single Fibre Modules

Single fibre modules were developed to optimise culture conditions for growth of cord blood CD34⁺ cells. These consist of a single hollow fibre, which was housed in the bottom of a polystyrene tissue culture dish. The ends of the fibre were glued to silastic inlet and outlet tubes so that the inside of fibres could be inoculated with cells. Modules were sterilised using ethanol and UV light irradiation Single fibre modules were inoculated with thawed 1-2×10⁸ cord blood CD34⁺ cells per ml. These cells were enriched from cord blood donations (Dr Marcus Vowels, Australian Cord Blood Bank) using MACS kits (Becton Dickinson, Australia), and cryopreserved before thawing.

After inoculation of the single fibre, silastic tubes were clamped using brass clips. The bottom of the tissue culture dish was filled with extracapillary media. The internal volume of the fibre was only 1.3 μL (π×100 μm²×4 cm) whereas the volume of the extracapillary media was 2 ml.

Therefore the extracapillary media was in excess (˜1500:1). The intracapillary cell concentration was calculated as follows:

$$\frac{Cell number}{Volume of fibre segment}$$

where Volume of fibre segment=πr²l
where r=100 microns and l is the length of the fibre segment which is counted.
Image Analysis and Cell Counting

The growth of cells inside the hollow fibres could be directly observed using an inverted microscope.

Cells were counted by digital capture (Pulnix CCD camera,™ 1001) and image analysis software (Wit 5. 1). Images of cells growing inside fibres and cell counts were taken on days 0, 2, 5 and 8 of culture. The cell concentration was calculated from the average of 4 images taken at different positions along the fibre.

Extra- and Intracapillary Media

Tables 1 and 2 show the constituents of the intra- and extracapillary media. Cells were cultured in serum-free media (StemPro media and nutrient supplement, Gibco) containing growth factors (IL-3, SCF, TPO and Flt-3 ligand @ either 20 or 100 ng/ml). Some of the constituents of intra- and extracapillary media were varied, depending on the aim of the experiment.

TABLE 1
Intracapillary media

| Additive | Concentration | | Dilution | [Final] | |
|---|---|---|---|---|---|
| 2-mecaptoethanol | 0.1 | M | 1:100 | 1 | mM |
| Sodium pyruvate | 100 | mM | 1:100 | 1 | mM |
| Kanomycin | 69 | mg/ml | 1.5:1000 | 100 | μg/ml |
| penicillin | 41 | mg/ml | 1.5:1000 | 62 | μg/ml |

| | | | | |
|---|---|---|---|---|
| Growth factors (IL-3, | — | — | 20 or 100 | ng/ml |
| SCF, Flt-3 ligand, TPO) | | | | |

| | | | |
|---|---|---|---|
| StemPro-34 nutrient | N/A | 2.6:100 | N/A |
| supplement | | | |

TABLE 2
Extracapillary media

| Additive | Concentration | | Dilution | [Final] | |
|---|---|---|---|---|---|
| 2-mecaptoethanol | 0.1 | M | 1:100 | 1 | mM |
| Sodium pyruvate | 100 | mM | 1:100 | 1 | mM |
| Kanomycin | 69 | mg/ml | 1.5:1000 | 100 | μg/ml |
| penicillin | 41 | mg/ml | 1:5:1000 | 62 | μg/ml |
| +/− Insulin | — | | — | 10 | μg/ml |
| +/− Growth factors | | | | 20 | ng/ml |
| (IL-3, SCF, Flt-3 ligand, | | | | | |
| TPO) | | | | | |

| | | | | |
|---|---|---|---|---|
| BSA or Dextran 70 or | — | — | 10 | mg/ml |
| StemPro-34 nutrient | | | | |
| supplement | | | | |

In the first experiment we determined the effect of attachment on the growth of cord blood CD34⁺, cells using poly-Lysine as an attachment factor. Poly-L-lysine (100 μg/ml) was physically adsorbed onto the lumenal surface of hollow fibre modules by incubation overnight at room temperature. Modules were washed with media the next day before injection of cells.

The second experiment examined the influence of substituting StemPro media supplement in the extracapillary media with either bovine serum albumin (BSA) or dextran (therapeutic grade, molecular weight 70,000) at the same osmotic pressure (equivalent to 10 mg protein per ml). The relative costs of BSA, Dextran or StemPro media is $1.56, $0.89 and $80.00 per 100 mls of media. Dextran may have regulatory advantages since it is approved for human infusion.

The third experiment examined the feasibility of not including growth factors or insulin in the extracapillary media. The cost of growth factors is approximately $A200 per 100 mls of media.

The final experiment examined the influence of intracapillary growth factor concentration on growth (20 ng/ml versus 100 ng/ml).

Results

Influence of Cell Attachment

Cord blood CD34⁺ cells attach transiently (1-2 days) when fibres are coated with poly-L-lysine. This results in an initial growth lag at 2 days, however there was no significant difference in the expansion of cord blood cells at day 5 or day 8 (FIG. 1).

The distribution of cells along the fibre was more uniform when cells were attached. Unattached cells tended to form clumps along the fibre. If the hollow fibre device is used to pre-enrich cord blood by attachment of CD34⁺ cells using immobilised antibody, it is likely that the effect on growth will not be significant.

It is important to note that cells grew well beyond the maximal density for tissue culture flask culture. Cells in hollow fibres grew to a density of 20-40×10⁶/ml (Table 3). Cord blood CD34⁺ cells grown in tissue culture flasks do not grow beyond 2×10⁶ cells/ml.

Effect of Growth Factor Concentration

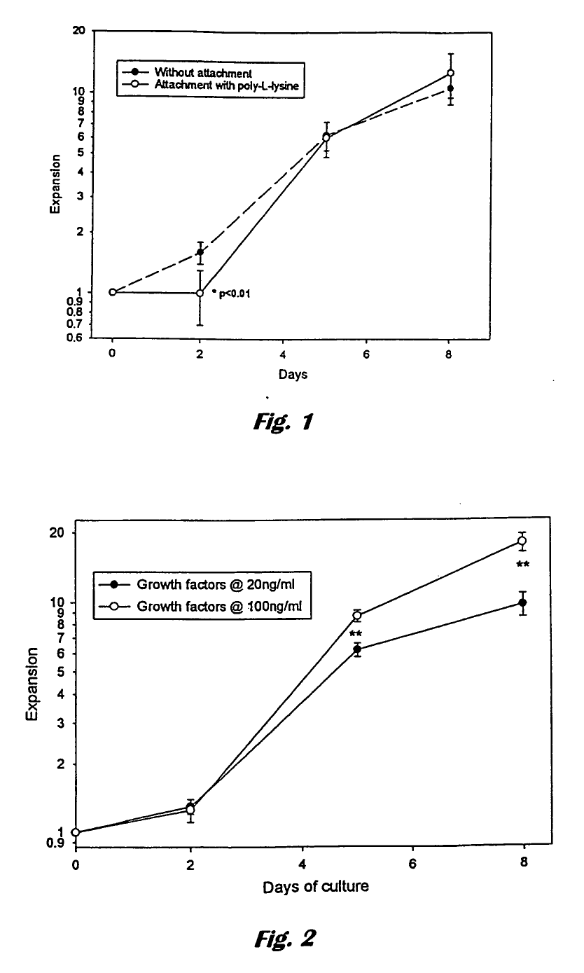

Removal of growth factors from the extracapillary media did not have an adverse effect on the growth of cord blood CD34⁺ cells. In contrast, the intracapillary concentration of growth factors had a marked influence on the expansion of cells. FIG. 2 shows that a fivefold increase in growth factor concentration (20 ng/ml versus 100 ng/ml) increased the number of cells by at least a factor of two. At higher cell concentrations there was multi-layer deposition of cells in hollow fibres, resulting in an underestimate of the cell number using image analysis software (FIG. 3).

A maximal concentration of 32±9×10⁸ cells/ml was reached at day 8 using growth factors at 100 ng/ml. It is estimated that cell numbers may be as much as twice this value because of multilayer cell deposition.

Influence of Insulin, Dextran or BSA in Extracapillary Media

The aim of these experiments was to determine whether cheaper alternatives to StemPro media supplement could be substituted into the extracapillary media. Fully-defined media such as StemPro contains additives such as human albumin, transferin, insulin, and low density lipoproteins. Since it possible that peptides such as insulin cross the membrane (molecular weight 8,000), it may be necessary to include insulin in the extracapillary media. It is also necessary to equalise the osmotic pressure caused by molecules greater than 5,000 molecular weight to prevent influx of water into the hollow fibres. This can be achieved using a molecule that does not cross the cellulose membrane (molecular weight>5,000). Alternatively, osmotic pressure may be equalised by appropriate selection of the pressure of the media on the extracapillary side.

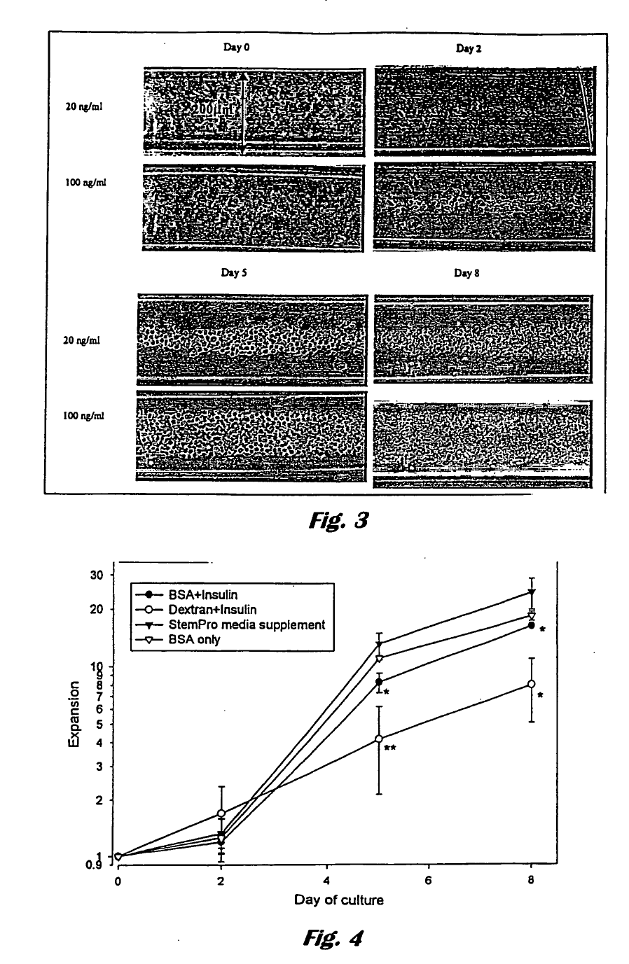

FIG. 4 shows the growth curves for this series of experiments. StemPro media supplement was the best extracapillary media, however BSA on its own could be substituted for StemPro media supplement with only a marginal (not statistically significant) decrease in growth. Surprisingly insulin in the extracapillary media appeared to reduce cell growth (p<0.05). There was poor growth and cell necrosis when dextran was substituted in the extracapillary media.

An important function of albumin is to bind calcium and small molecules such as insulin. Dextran does not bind these molecules, and it may be necessary to adjust calcium so that intracapillary free calcium levels are in the physiological range.

### EXAMPLE 2

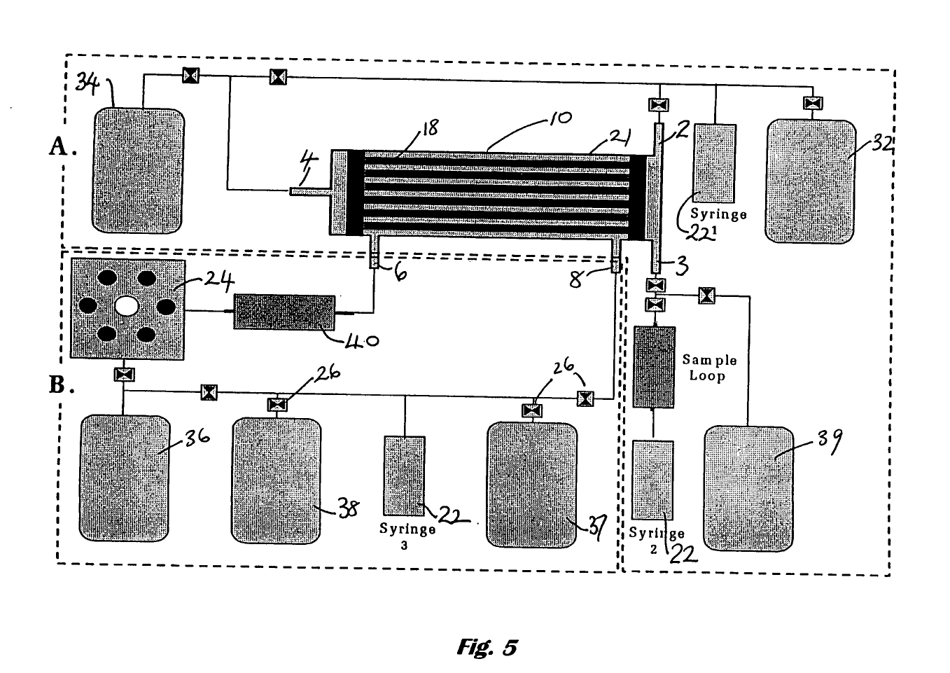

An embodiment of a bioreactor of the invention, in the form of a bioreactor, is shown in FIG. 5. The bioreactor is designed for combined cell selection and expansion. The necessary components for cell loading and harvesting, or perfusion cultures are shown in boxes A and B, respectively. An autoclavable hollow fibre bioreactor module 10 is housed inside a purpose-built incubator (42×40×47 cm) that controls environmental variables for high-density, perfusion culture (media perfusion rate, temperature & CO₂) in addition to cell selection processes. The incubation chamber in this case maintains temperature at 37° C. and CO₂ at 5%.

Hollow cellulose fibres 18 are housed within a cylindrical shell 21 of the module using the standard kidney dialyser configuration. A medium-scale hollow fibre module is one that can be used to produce 10⁸ cells/200 cm² and a large-scale cellulose hollow fibre module may be one suitable for producing 10¹⁰ cells/m².

The bioreactor module housing 21 has inlet ports 2 and 3 for introduction of intracapillary media and cells respectively and outlet port 4 for removal of intracapillary waste. The housing also has inlet and outlet ports 6 and 8 for introduction of extracapillary media and extracapillary waste respectively. The device includes a gas and heat exchanger 40 through which passes the extracapillary media.

Intracapillary media and waste are stored in containers 32 and 34 respectively. Extracapillary media and waste are stored in containers 36 and 38 respectively. Cells are stored in container 39.

Flow control in the device is achieved by stepper motor driven syringe pumps 22 and a peristaltic pump 24 interfaced with an IBM compatible PC (not shown). Fluid paths through the unit are controlled by solenoid pinch valves 26 also interfaced with the IBM compatible PC. The module is rotated by a stepper motor driven turntable (not shown).

The system is controlled using Labview™ software running on the IBM compatible PC. This software platform provides the tools to create a “virtual instrument” interface which monitors and controls all aspects of devices operation (pump speeds, flow paths, reservoir volumes, temperature and CO₂). A graphical user interface is used to program and automate the cell separation and culture process.

The system is easily scalable to process from 10⁸ to 10¹⁰ cells using already established cellulose renal dialyser technology. These devices are approved for extra-corporeal use (direct contact with blood), which greatly simplify regulatory approval.

An advantage of this embodiment of the bioreactor of the present invention is the integration of cell separation and culture as a single platform and growth of cells in the intracapillary space. Because cells are processed within a closed sterile environment and cell handling is automated, the device is ideally suited for processing of cells under Good Manufactureing Practice (GMP). Other advantages include the ease of scale-up using the compact configuration of a hollow fibre dialyser (1-2×10¹⁰ cells per module) as well as potential cost savings associated with reduced consumption of growth factors and albumin (see below).

In use, the hollow fibres 18 are internally coated with an antibody. For example, to expand CD34⁺ cells, the hollow fibres are internally coated with anti-CD34 moAb. After capture of CD34⁺ cells from mononuclear cell concentrates, the cells are expanded inside hollow fibres by perfusion of the extracapillary space with tissue culture media.

Cells are injected into the module via port 3 by drawing fluid from port 4 followed by injection of fluid by port 2 to wash remaining cells from the header of the module into hollow fibres. Ports 2, 3 and 4 are sealed by closure of valves leading to these ports, and the module rotated for 30 minutes whilst cells attach to hollow fibres. Unbound cells are washed out of the module 10 at low shear stress (10-25 dynes/cm²) into reservoir 39.

Cells are cultured for 1 to 2 weeks by pumping media (pump 24) through the module via the extracapillary circuit (ports 6 and 8). The flow rate is approximately 2 ml/hour/10⁶ cells. Media is replaced at regular intervals by pumping media from reservoir 36 into reservoir 38, and replenishing the media from the media refill reservoir. During the culture period it may be necessary to inject intracapillary media from reservoir 32 at a much slower rate (less than 1 ml/10⁸ cells/day).

At the conclusion of culture cells are harvested by injecting media at a rate which will displace cells out of hollow fibres (>10 dynes/cm²) into collection bags (reservoir or cell bag 39). Enzymes, EDTA or other cell releasing agents may be required to detach cells which are still bound.

The device described above has been tested using mobilised peripheral blood and has similar or superior performance to other CD34⁺ cell selection technologies (Enrichment 1200-fold, Yield 61%).

### EXAMPLE 3

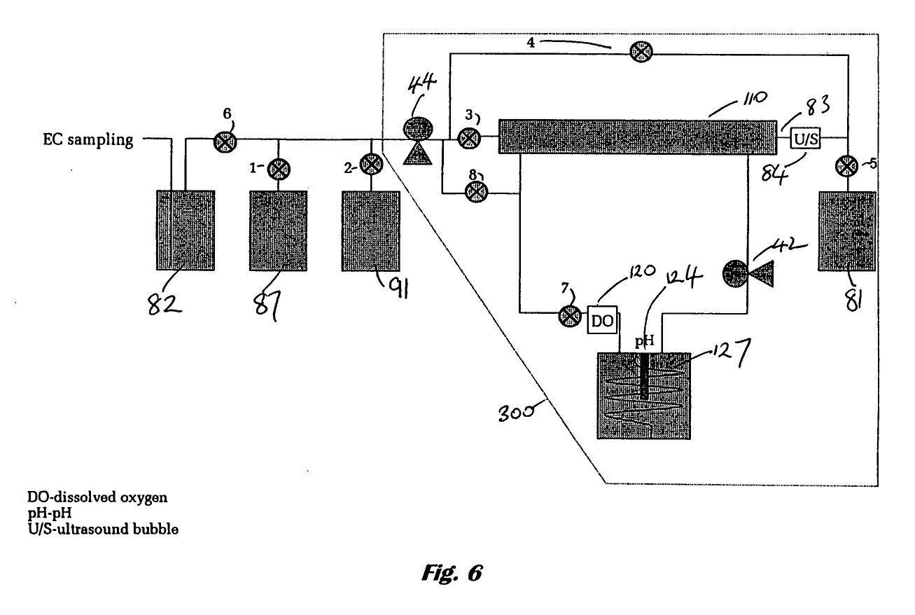

A further embodiment of a bioreactor in accordance with the present invention is shown in FIG. 6. Typical components of this embodiment are given in Table 4. The main physical requirement is that the system be portability (<20 kg) and size (<300 mm height×<450 mm width×<450 mm depth). The system has a removable plastic hood (not shown), which is dark brown to filter UV light, enclosing the incubator area.

Referring first to FIG. 6, one or more modules 110 containing cellulose hollow fibre capillaries in cylindrical housing(s) are used to separate and grow cells. CD34⁺ cells contained in cells loaded from cell reservoir 81 via inlet port 83 are captured onto the inner surface of hollow fibres by immobilised monoclonal antibodies, linked to the cellulose substrate with a cellulose-binding domain. An ultrasonic bubble detector 84 positioned between the cell reservoir and the inlet port 83 of the module assists in the loading of cells into the module. Cells are drawn into the module until the bubble detector senses a gas interface.

Enriched cells were left in situ, and the extracapillary space was perfused with acellular culture media from reservoir 127 via acellular inlet port 89. There may be intracapillary flow of media from intracapillary media reservoir 91 via intracapillary inlet 83 as well. Cells were harvested after 1-3 weeks of growth using fluid shear and cell releasing agents if necessary.

The molecular weight cut off of the cellulose membrane is greater than 10,000. Therefore it is possible to decouple the supply (and removal) of low and high molecular weight components of the culture media. The consumption of albumin and growth factors are minimised by not including them in the extracapillary media, which supplies all low molecular weight substrates (glucose, oxygen, amino acids etc).

Solenoid pinch valves (1-8) control fluid paths. Two peristaltic pumps 42, 44 control extracapillary and intracapillary flow respectively. Table 5 shows the typical valve and pump states of the bioreactor of this embodiment.

There is communication between the extracapillary and intracapillary circuits via valve 8 so that higher flow rate may be supplied to the intracapillary circuit for cell harvesting. This communication also facilitates the removal of degraded extracapillary media to waste 82 and its replenishment with fresh media without interruption of the flow of conditioned-media during the culture process.

Oxygen uptake of the biomass was used to control the extracapillary perfusion rate. Oxygen uptake of the biomass was measured on the extracapillary circuit with oxygen sensor 120 in the form of polarographic electrode by comparison of upstream (clockwise recirculation) and downstream (anticlockwise recirculation) measurements. The metabolic uptakes of oxygen, glucose and lactate were measured using the system and are shown in Table 3. The extracapillary flow rate will need to keep up with these rates and prevent significant depletion or accumulation of these components.

TABLE 3
Specific metabolic uptake and production rates (mole/cell/sec)

| Cell type | Oxygen | Glucose | Lactate |
|---|---|---|---|
| KG1a | −1.3 × 10⁻¹⁷ | −3.0 × 10⁻¹⁶ | +3.5 × 10⁻¹⁶ |
| Expanding CB | −1.3 × 10⁻¹⁷ | — | — |

A bicarbonate/HEPES buffer system was used to control pH and an in-line pH electrode 124 was used to monitor pH.

Gases were exchanged with extracapillary media using a length of silastic tube 127 through which the gas is passed. The gas mixture used was carbon dioxide (5%) oxygen (5-20%) and nitrogen (75-90%).

The bioreactor system is enclosed and maintained at a temperature of 37±0.1° C. using the heater and fan elements (not shown). The dotted region 300 defines which components are held at 37° C. It will also be necessary to pressurise fluid within the system to 100 mm Hg to prevent the formation of gas bubbles inside the module and tubing.

The module itself has a surface area of 200 cm² (small-scale) or 1.4 m² (large-scale). The fibre diameter is 200 μm, and the module length is 30 cm.

TABLE 4
Specification of Cell System components

| Component | Tasks and comments |
|---|---|
| Embedded PC | TTL outputs for two stepper motors, 10 solenoid |
| | valves, duty cycle heater element and control of |
| | other systems. |
| | TTL inputs/outputs for bubble detector |
| | A-D conversion for two oxygen probes, one pH probe |
| | and up to 4 thermistors |
| | D-A conversion for DC motor controller |
| | Ethernet connection |
| | Interfaces - graphics, keyboard, serial port |
| Interface | Stepper motor drivers |
| board(s) | Solenoid valve drivers |
| | DC motor controller |
| | Heater block driver |
| | Input output board for A-D, D-A, TTL in/out if |
| | not included in the embedded PC |
| | Amplifiers for all analogue devices (thermistors, bubble |
| | detection, polarographic electrodes, pH electrode) |
| | DC motor controller |
| Software | Portable drivers or Vl's for each physical component |
| | Client graphical user interface (assuming communication |
| | is via TCP/IP and/or a serial port) |
| | Facility to program and control all hardware elements |
| | using a user friendly GUI. |
| | Error detection and diagnostics |
| Pumps | Stepper motor driven pump for intracapillary flow control |
| | (0.002-0.2 ml/min small-scale, 0.1-10 ml/min large-scale) |
| | DC motor driven pump for extracapillary flow control |
| | (0.21-21 ml/min small-scale, 3.8-380 ml/min large-scale) |
| Solenoid | Eight normally closed solenoid pinch valves, 2 |
| valves | normally open solenoid pinch valves |
| Polarographic | Measure oxygen level |
| electrodes | |
| pH Electrode | In direct contact with culture media via a flow cell. |
| Gas exchange | Small-scale: Single silastic tube inside of sealed metal |
| module | box which is convected with gas mixture |
| | Large-scale: As above or a polypropylene gas exchange |
| | hollow fibre module. |
| U/S detector | Ultrasonic probe to detect entry of gas into tube |
| Tubing, | Ultrasonic probe to detect entry of gas into a tube. |
| Fittings and | Medical or Pharmaceutical grade (e.g., PharMed ™⁾ |
| containers | |
| Hollow fibre | Cuprophan. Ethylene oxide sterilised |
| modules | Small-scale: Custom built by AKSO (200 cm²) |
| | Large-scale: Standard renal dialyser (0.8-1.4 m²) |
| Metal frame | Corrosion resistant/stainless steel drip tray to |
| | catch fluid spills. |

TABLE 5
Typical valve and pump states

| | IC | EC | | | | | | | | |
|---|---|---|---|---|---|---|---|---|---|---|
| Process | pump | pump | 1 | 2 | 3 | 4 | 5 | 6 | 7 | 8 |
| Power off | | | c | c | c | c | c | c | c | c |
| Draw cells | | cw | c | c | o | c | o | c | | c |
| Fill sample tube | acw | | c | o | c | o | o | c | | c |
| Concentrate cells | acw | | c | o | c | o | c | c | | c |
| in module | | | | | | | | | | |
| Perfuse cells | cw | | o | c | o | c | o | c | | c |
| with IC media | | | | | | | | | | |
| Load recirculated | acw | acw | o | c | c | c | c | c | o | o |
| IC media | | | | | | | | | | |
| Empty recirculated | cw | acw | c | c | c | c | c | o | o | o |
| EC media reservoir | | | | | | | | | | |
| Recirculate EC | | acw | | | | | | | o | c |
| media | | | | | | | | | | |
| Draw volume into | | cw | | | | | | | c | c |
| EC space | | | | | | | | | | |

(cw = clockwise, acw = anticlockwise, c = closed, o = open, EC = extracapillary, IC = intracapillary)

### EXAMPLE 4

Growth of KG1a Cells using Perfusion Bioreactor

A hollow fibre cellulose minidialyser (intracapillary volume=1 ml) was used to grow KG1a cells using the bioreactor system depicted in FIG. 5. Media was recirculated on the extracapillary side of the module, whilst there was no intracapillary flow.

Aims

1. Determine “baseline” performance of the system using a tumour cell line, KG1a cells
2. Measure glucose and oxygen uptake, and lactate production.
3. Compare growth rate with flask tissue culture control.
   Methods

Table 6 shows the bioreactor operating parameters for this series of experiments. All bioreactor runs had the same oxygen tension, pC0₂, and media additives (foetal calf serum). The variable that was different between runs was the extracapillary perfusion rate and the number of cells inoculated.

TABLE 6
Perfusion bioreactor operation

| Control variables | Run 4 | Run 5 | Run 8 |
|---|---|---|---|
| Oxygen | 20% | 20% | 20% |
| CO₂ | 5% | 5% | 5% |
| Base media | DME | DME | RPMI |
| IC media additives | 50% FCS | 50% FCS | 50% FCS |
| EC media additives | 10% FCS | 10% FCS | 10% FCS |
| IC flow rate | 0 | 0 | 0 |
| EC flow rate | 0.9 ml/min | 0.9-3.2 ml/min | 0.2 ml/min |
| Input | 3 × 10⁶ KG1a | 14.5 × 10⁸ KG1a | 5 × 10⁶ KG1a |
| | cells | cells | cells |
| EC volume | 80 ml | 80 ml | 80 ml |
| IC volume | 1 ml | 1 ml | 1 ml |

Results

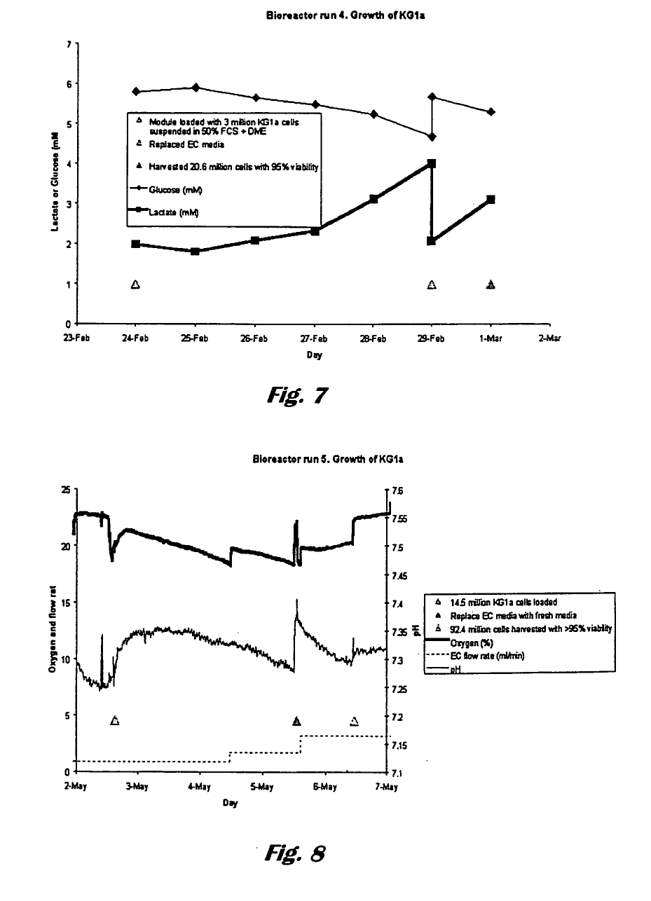

FIG. 7 shows the glucose and lactate levels in EC media during the bioreactor run. EC media was replaced on the 29^{th} February, resulting in a depletion of glucose and an increase in lactate. KG1a cells were harvested after 6 days of growth (>95% viability) and had increased by a factor of 6.9 (average doubling time=52 hours)

In run 5 it was possible to measure EC oxygen concentration and pH at the outlet of the hollow fibre module. FIG. 8 shows these measurements as well as the EC flow rate.

The oxygen ramps down and can be increase by increasing EC perfusion rate. The pH was decreasing on day 2 and 3 (4^{th}-5^{th} May), and so the media was replaced on 6^{th} May. After harvest of the cells, the EC oxygen concentration increased in a stepwise fashion because there was no longer a biomass to consume oxygen. KG1a cells were harvested after 4 days of growth (>95% viability) and had increased by a factor of 6.4 (average doubling time=35 hours).

The results of run 8 are summarised in Table 7. In this experiment an accurate growth rate was estimated by inoculating the module with 10 micron polystyrene beads with the cells and determining the ratio of beads to cells by flow cytometry before and after expansion. In this case the cells grew at a similar rate to run 5 (doubling time=35 hours) and static tissue culture controls.

TABLE 7
Run 8

| Number of inoculated cells | 5 million cells |
|---|---|
| Bead to cell ratio at inoculation | 1:19.8 |
| Number of harvested cells | 14.3 million |
| Bead to cell ratio at 3 days | 1:81 |
| Fold expansion (bioreactor) | 4.1 |
| Fold expansion (tissue culture flask) | 3.4 |

### CONCLUSIONS

The perfusion bioreactor has the capacity to grow KG1a cells at concentrations that are at least 90 times higher than in static tissue culture flasks (90×10⁶ cells/ml versus 10⁶ cells per ml). The rate of growth achieved was similar to static flask culture, and is likely to depend on the rate of extracapillary perfusion. If the perfusion rate is low, then oxygen depletion may occur near the outlet of the bioreactor. Conversely, run 4 demonstrated that high perfusion rates may be detrimental to the growth of KG1a cells since KG1a cells did not grow as well at “low” cell density and relatively high perfusion rate. Growth rate was improved by lowering the extracapillary perfusion rate (run 8=0.2 ml/min versus run 4=0.9 ml/min).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for culturing one or more type(s) of cells, the method comprising:
providing a semi-permeable substrate having the cells on one side thereof (cellular side), wherein the semi-permeable substrate is permeable to at least one substance selected from the group consisting of a nutrient, a regulator and a metabolite, but is substantially impermeable to at least one protein required for proliferation, differentiation and/or genetic modification of the cells;
contacting the cells with a culture medium comprising at least one protein required for proliferation, differentiation and/or genetic modification of the cells, and optionally at least one substance required for the proliferation of the cells; and
providing on the acellular side of the semi-permeable substrate at least one substance required for proliferation of the cells.

2. A process according to claim 1 wherein the at least one substance required for proliferation of the cells is contained in media perfusing over at least a part of the acellular surface of the semipermeable membrane.

3. A method according to claim 2 wherein the acellular media is recirculated to the semipermeable substrate.

4. A method according to claim 2 wherein the acellular media perfusion rate is responsive to the cellular biomass.

5. A method according to claim 4 wherein the biomass is determined by measuring oxygen uptake, glucose uptake and/or lactate output in the cellular media.

6. A method according to claim 5 wherein the perfusion rate is determined by oxygen uptake.

7. A method according to claim 4 wherein perfusion rate is controlled so as to prevent significant depletion or accumulation of the at least one substance required for proliferation of the cells and/or waste products in the acellular space.

8. A method according to claim 3 wherein the acellular media is replaced at a preselected rate.

9. A method according to claim 8 wherein the preselected rate is less than or equal to approximately 2 ml/hr/10⁶ ml.

10. A method according to claim 1 wherein the semi-permeable substrate is impermeable to molecules having a molecular weight at least about 10,000.

11. A method according to claim 10 wherein the semi-permeable substrate is impermeable to molecules having a molecular weight of at least 8,000.

12. A method according to claim 10 wherein the semi-permeable substrate is impermeable to molecules having a molecular weight 5000.

13. A method according to claim 1 wherein the semi-permeable substrate is in the form of at least one hollow fibre.

14. A method according to claim 13 wherein the hollow fibres have a radius in the range of about 100 to 400 microns and a wall thickness in the range of about 6 to 50 μm.

15. A method according to claim 12 wherein the hollow fibres are formed from a semipermeable material selected from the group consisting of cellulose, cellulose acetate and polysulfone.

16. A method according to claim 15 wherein the hollow fibres are formed from cellulose.

17. A method according to claim 1 wherein the cells are bound to the semi-permeable substrate by at least one ligand.

18. A method according to claim 15 wherein the ligand is selected from the group consisting of an antibody, lectin, growth factor and receptor.

19. A method according to claim 18 wherein the ligand is an antibody.

20. A method according to claim 19 wherein the ligand is a monoclonal antibody.

21. A method according to claim 1 wherein the cells are selected from the group consisting of animal cells, plant cells, fungi cells and microorganisms.

22. A method according to claim 1 wherein the cells are mammalian cells.

23. A method according to claim 1 wherein the cells are selected from the group consisting of haematopoietic cells (GD34⁺), T cells, B cells, dendritic cells, liver cells, bone marrow cells, pancreatic islet cells, embryonic stem cells and genetically modified cells.

24. A method according to claim 23 wherein the cells are chinese hamster ovary (CHO) cells or hybridomas.

25. A method according to claim 1 wherein the cells are in a coculture system.

26. A method according to claim 1 wherein the at least one protein required for cell proliferation, differentiation and/or genetic modification is selected from one or more of the group consisting of growth factors, colony stimulating factors, cytokines, cytokine receptors, chemokines, albumin, transferring, low density lipoproteins, and gene transfer vectors.

27. A method according to claim 26 wherein the at least one protein required for cell proliferation, differentiation and/or genetic modification is at least one growth factor selected from one or more of the group consisting of IL-1, IL-2, IL-3, SCF, IL-6, Flt-3 ligand, insulin, thrombopoietin, erythropoietin, EGF, TNF, TGFβ, PDGF, NGF, and FGF.

28. A method according to claim 26 wherein the at lease one protein required for cell proliferation, differentiation and/or genetic modification is GCSF or GMCSF.

29. A method according to claim 29 wherein the chemokine is selected from the group consisting of MIPIα, SDF-1 and insulin-like growth factor.

30. A method according to claim 26 wherein the gene transfer vectors are selected from the group consisting of non-replicative retroviral and adeno-associated viral vectors, lipoplexes and phage vectors.

31. A method according to claim 1 wherein the at least one substance required for proliferation is selected from the group consisting of glucose, amino acids, vitamins and steroid hormones.

32. A method according to claim 1 the cells are of a desired cell type separated from a sample comprising the desired cell types.

33. A method according to claim 32 wherein the cells of a desired cell type are removed from a sample containing the desired cells by loading the sample into a device comprising a semi-permeable substrate provided with a ligand reactive with the desired cell type, incubating to allow deposition and binding of the desired cell type to the ligand, treating the semi-permeable substrate in a manner such that the cells not bound to the ligand are removed, and optionally treating the semipermeable substrate in a manner such that the cells not bound to the ligand are removed.

34. A method according to claim 32 wherein the cell separation and cell culture are carried out in a single bioreactor.

35. A method according to claim 1 used for expansion of the cells.

36. A method according to claim 35 wherein the cells are used to generate neutrophil and platelet precursors.

37. A method according to claim 36 wherein the neutrophil and platelet precursors are generated by stimulating haematopoietic stem cells (CD34⁺) to proliferate and differentiate with haematopoietic growth factors.

38. A method according to claim 36 wherein the cells are in a coculture system used to generate haematopoietic cells.

39. A method according to claim 38 wherein a bone marrow stromal cell layer is established within the hollow fibres that supports the growth of haematopoietic stem cells.

40. A method according to claim 35 wherein cytotoxic T cells are generated by T cell receptor engagement and crosslinking.

41. A method according to claim 40 where the cellular media contains IL-2 and B7-1 and B7-2 molecules found on antigen presenting cell.

42. A method according to claim 35 wherein the cells are antigen-specific T cell clones in a coculture system.

43. A method according to claim 42 wherein the coculture system uses a monolayer of cells selected from the group consisting of dendritic cells, monocytes or fibroblasts.

44. A method according to claim 35 wherein the cells are haematopoietic or immune cells that are transduced by at least one retroviral gene transfer vector in the cellular media.

45. A bioreactor for the proliferation and growth of cells, the bioreactor comprising
a plurality of hollow fibres for containment of cells therein and formed from a semipermeable material that is permeable is permeable to at least one substance selected from the group consisting of a nutrient, a regulator and a metabolite but is substantially impermeable to at least one protein required for proliferation, differentiation and/or genetic modification, the hollow fibres being positioned within a housing defining an acellular space;
housing inlet and housing outlet means communicating through the acellular space to define an acellular flow path;
a liquid flow circuit providing fluid communication between the housing inlet and outlet means; and
circulation means associated with the liquid flow circuit to circulate media through the acellular space, the circulation means being responsive to the cell biomass.

46. A bioreactor according to claim 45 wherein the hollow fibres contain cells and at least one protein required for proliferation, differentiation and/or genetic modification of the cells in the lumen thereof.

47. A bioreactor according to claim 45 wherein the acellular space contains media comprising at least one substance required for proliferation of the cells.

48. A bioreactor according to claim 47 wherein the at least one substance is selected from the group consisting of glucose, amino acids, vitamins, steroid hormones and mixtures of two or more thereof.

49. A bioreactor according to claim 45 wherein the hollow fibres are formed from a semi-permeable material selected from the group consisting of cellulose, cellulose acetate and polysulfone.

50. A bioreactor according to claim 49 wherein the semi-permeable material is cellulose.

51. A bioreactor according to claim 50 wherein the hollow fibres have a diameter of about 100 to 400 μm and a wall thickness in the range of about 6 to 50 μm.

52. A bioreactor according claim 45 wherein the circulation means is at least one pump.

53. A bioreactor according to claim 45 wherein the cellular biomass is determined measuring means for measuring oxygen uptake, metabolite uptake and/or lactate output.

54. A bioreactor according to claim 53 wherein the biomass measuring means determines oxygen uptake in the acellular media.

55. A bioreactor according to claim 45 further comprising gas control means for controlling oxygen and carbon dioxide content of the acellular media.

56. A bioreactor according to claim 55 wherein the gas control means is gas exchange means.

57. A bioreactor according to claim 56 wherein the gas exchange means comprises a silicone membrane.

58. A bioreactor according to claim 57 wherein the gas exchange means is a silicone tube in fluid communication with the liquid flow circuit and passing through a gas chamber.

59. A bioreactor according to claim 45 further comprising means to control the temperature of media flowing in the liquid flow circuit.

60. A bioreactor according to claim 45 wherein the liquid flow circuit recycles the acellular media to the acellular space.

61. A method according to claim 45 further comprising means for replacing the acellular media with fresh media at a preselected rate.

62. A bioreactor according to claim 45 wherein the hollow fibres are provided internally with at least one ligand.

63. A bioreactor according to claim 62 wherein the ligand is selected from the group consisting of an antibody, lectin, growth factor and receptor.

64. A bioreactor according to claim 63 wherein the ligand is an antibody.

65. A bioreactor according to claim 64 wherein the ligand is a monoclonal antibody.

66. A bioreactor according to claim 45 wherein the cells are selected from the group consisting of animal cells, plant cells, fungi cells and microorganisms.

67. A bioreactor according to claim 66 wherein the cells are mammalian cells.

68. A bioreactor according to claim 67 wherein the cells are selected from the group consisting of haematopoietic cells (CD34⁺), T cells, B cells, dendritic cells, liver cells, bone marrow cells, pancreatic islet cells, embryonic stem cells or genetically modified cells such as chinese hamster ovary (CHO) cells and hybridomas.

69. A bioreactor according to claim 45 wherein the bioreactor is capable of both cell separation and cell culture.

70. A bioreactor according to claim 45 which is portable.
